(19) United States
(12) Patent Application Publication
(10) Pub. No.: US 2010/0049023 A1
(43) Pub. Date: Feb. 25, 2010

(54) Non-invasive method and system for determining physiological characteristics

(76) Inventors: Bernhard B. Sterling, Danville, CA (US); Andrew R. Lawrence, Eagan, MN (US); Gregory I. Voss, Solana Beach, CA (US); James M. Perry, Nashville, TN (US); Rankin A. Clinton, III, Franklin, TN (US)

Correspondence Address: Ralph C. Francis;Francis Law Group, 1942 Embarcadero, Oakland, CA, 94606, US

(21) Appl. No.: 12/229,158

(22) Filed: Aug. 20, 2008

(51) Int. Cl. A61B 5/1491

(52) U.S. Cl. 600/363

(57) ABSTRACT

A non-invasive method of determining a physiological characteristic, comprising providing at least one physiological sensor that is adapted to measure at least one physiological characteristic at a target measurement site on a subject's body, heating an extended tissue region on the subject's body, whereby blood perfusion of the tissue region is enhanced, and measuring at least one physiological characteristic with the physiological sensor during or within a predetermined period after heating the extended tissue region.

### FIELD OF THE PRESENT INVENTION

The present invention relates to the field of pulse oximetry. More specifically, the invention relates to a pulse oximetry method and system that employs heating means to enhance blood perfusion.

### BACKGROUND OF THE INVENTION

It is well known in the art that pulse oximetry is based on the principle that the color of blood is related to the oxygen saturation level of hemoglobin. Indeed, as blood deoxygenates, the pinkish skin color (in many individuals) transitions to a bluish hue. This phenomenon allows measurements of the degree of oxygen saturation of blood using, what is commonly referred to as, optical pulse oximetry technology.

Pulse oximetry devices, i.e. oximeters, typically measure and display various blood constituents and blood flow characteristics including, blood oxygen saturation of hemoglobin in arterial blood, the volume of individual blood pulsations supplying the flesh and the rate of blood pulsations corresponding to each heartbeat of the patient. Illustrative are the devices disclosed in U.S. Pat. Nos. 5,193,543, 5,448,991, 4,407,290 and 3,704,706.

As is well known in the art, a pulse oximeter passes light through human or animal body tissue where blood perfuses the tissue, such as a finger or ear, and photoelectrically senses the absorption of light in the tissue. Since oxygenated and deoxygenated hemoglobin absorb visible and near infrared light differently, two lights having discrete frequencies in the range of about 650-670 nm in the red range and about 800-1000 nm in the infrared range are typically passed through the tissue. The amount of transmitted light passed through the tissue varies in accordance with the changing amount of blood constituent, i.e. oxygen (or oxygen saturation), in the tissue and the related light absorption.

Two oxygen saturation parameters can readily be ascertained via oximetry; arterial oxygen saturation and peripheral, arterial oxygen saturation. Arterial oxygen saturation (SaO₂) is based on direct measurement of light absorption in tissue and/or blood based on all commonly measured hemoglobin components. Peripheral, arterial oxygen saturation (SpO₂), as measured by pulse oximetry, is generally determined by measuring the constant (non-pulsatile) and pulsatile light intensities (discussed below) of the two functional components oxyhemoglobin and deoxyhemoglobin, at each of the two noted wavelengths, and correlating the measured intensities to provide peripheral oxygen saturation.

As is also well known in the art, variations in tissue temperature proximate the measurement site can, and in many instances will, affect blood perfusion and, hence, oximetry measurements dependant thereon. Indeed, a rise in tissue temperature induces or triggers a homeostatic reflex, which enhances local blood flow in order to increase the transfer of heat away from the skin. The enhanced blood flow or perfusion will enhance the accuracy of the oximetry measurement, since the light transmitted to the tissue will encounter a larger volume of blood.

Various heating means have thus been incorporated in sensors or pulse oximeters to improve blood perfusion adjacent the sensor. Illustrative are the sensors disclosed in U.S. Pat. Nos. 4,926,867, 5,299,570, 4,890,619 and 5,131,391.

In U.S. Pat. No. 4,926,867, an oximeter sensor is disclosed that includes a metal plate that functions as a heater. According to the invention, the heater is adapted and positioned to heat the tissue proximate the sensor to enhance blood perfusion. A separate thermistor is also provided to monitor the amount of heat transmitted to the tissue by the heater.

U.S. Pat. Nos. 5,299,570 and 4,890,619 disclose oximeter sensors that employ ultrasonic energy to enhance blood perfusion. The blood perfusion is similarly enhanced primarily proximate the sensor.

Various substances have also been applied to the skin (or tissue site) to enhance blood perfusion. Illustrative are the pulse oximeter methods disclosed in U.S. Pat. Nos. 5,392,777, 5,267,563 and 6,285,896.

In U.S. Pat. Nos. 5,392,777 and 5,267,563, a counterirritant is applied to the skin prior to attachment of the oximeter sensor. In U.S. Pat. No. 6,285,896, a vasodilating substance is applied to the skin prior to attachment of the oximeter sensor to reduce the effects of localized oxygen consumption and to increase blood fraction.

Although the noted sensor systems and methods provide effective means to enhance blood perfusion, there are a number of disadvantages and drawbacks associated with the systems and methods. A major drawback is that the enhanced blood perfusion realized by the conventional sensor systems and methods is typically localized, i.e. proximate the sensor. As discussed in detail herein, applicants have found that the signal-to-noise ratio of an oximeter sensor (and, hence, the accuracy of any physiological characteristic, e.g., O₂ saturation, determined therefrom) can be significantly enhanced by heating an entire organ or appendage, e.g., ear or hand, prior to or in conjunction with taking an oximeter reading.

A further drawback is that virtually all of the conventional sensor heating means comprise means for heating the sensor (or housing thereof) or a member that is integral thereto, e.g., heated plate. Such heating means necessitates frequent site changes to avoid thermal injury, which makes the monitoring method (employing the heating means) more labor intensive and costly than other non-invasive monitoring methods.

Additional drawbacks are that the conventional sensor systems and methods require extensive and complex sub-systems to regulate the amount of heat transmitted to the skin site and avoid burning the patient, and are typically limited to one sensor and, hence, one sensor location on the body.

It would therefore be desirable to provide a simple physiological sensor method and system that substantially reduces or overcomes the disadvantages and drawbacks associated with conventional sensor methods and systems, such as pulse oximeter sensor methods and systems.

It is therefore an object of the invention to provide a physiological sensor method and system that substantially reduces or overcomes the disadvantages and drawbacks associated with conventional sensor methods and systems.

It is another object of the invention to provide a physiological sensor method and system that enhance the accuracy of physiological measurements and determinations made therefrom.

It is another object of the invention to provide a pulse oximetry method and system that includes heating means to enhance blood perfusion.

It is another object of the invention to provide a pulse oximetry method and system that includes heating means that is adapted to heat a significantly larger tissue region, such as an entire ear or hand, prior to or in conjunction with obtaining an oximeter reading therein.

It is another object of the invention to provide a pulse oximetry method and system that includes multiple sensors and associated heating means that are adapted to selectively heat a large tissue region.

### SUMMARY OF THE INVENTION

In accordance with the above objects and those that will be mentioned and will become apparent below, in one embodiment of the invention, there is provided a non-invasive method of determining a physiological characteristic, comprising the steps of (i) providing at least one physiological sensor that is adapted to measure at least one physiological characteristic at a target measurement site on a subject's body, (ii) disposing the physiological sensor proximate the target measurement site, (iii) heating an extended tissue region on the subject's body, whereby blood perfusion of the tissue region is enhanced, the extended tissue region including the target measurement site and a region extending beyond the target measurement site, and (iv) measuring at least one physiological characteristic with the physiological sensor during or within a predetermined period after heating the extended tissue region.

Preferably, heating of the extended tissue region is sufficient to induce or trigger an optimal homeostatic reflex, whereby tissue perfusion is enhanced, without burning the subject.

In one embodiment of the invention, the extended tissue region comprises the entire ear of the subject and the target measurement site comprises the earlobe of the heated ear.

In another embodiment, the extended tissue region comprises the entire ear and adjoining structure, i.e. tissue of the head adjacent the ear, of the subject and the target measurement site comprises the earlobe of the heated ear and adjoining structure.

In another embodiment of the invention, the extended tissue region comprises the entire arm of the subject and the target measurement site comprises a finger on the heated arm.

In yet another embodiment, the tissue region comprises a hand of the subject and the target measurement site comprises a finger on the heated hand.

In one embodiment of the invention, the physiological characteristic comprises the blood oxygen saturation of the subject.

In accordance with another embodiment of the invention, there is provided a non-invasive method of determining a physiological characteristic, comprising the steps of (i) providing a plurality of physiological sensors that are adapted to measure at least one physiological characteristic at target measurement sites on a subject's body, (ii) disposing a first physiological sensor proximate a first target measurement site on the subject's body and a second physiological sensor proximate a second target measurement site on the subject's body, (iii) heating a first extended tissue region on the subject's body, whereby blood perfusion of the first extended tissue region is enhanced, the first extended tissue region including the first target measurement site and a region extending beyond the first target measurement site, and (iv) measuring at least one physiological characteristic with the first and second physiological sensors during or within a predetermined period after heating the first extended tissue region.

In accordance with another embodiment of the invention, there is provided a non-invasive method of determining a physiological characteristic, comprising the steps of (i) providing a plurality of physiological sensors that are adapted to measure at least one physiological characteristic at target measurement sites on a subject's body, (ii) disposing a first physiological sensor proximate a first target measurement site on the subject's body and a second physiological sensor proximate a second target measurement site on the subject's body, (iii) heating a first extended tissue region on the subject's body, whereby blood perfusion of the first extended tissue region is enhanced, the first extended tissue region including the first target measurement site and a region extending beyond the first target measurement site, (iv) heating a second extended tissue region on the subject's body, whereby blood perfusion of the second extended tissue region is enhanced, the second extended tissue region including the second target measurement site and a region extending beyond the second target measurement site, and (v) measuring at least one physiological characteristic with the first and second physiological sensors during or within a predetermined period after heating the first and second extended tissue regions.

In accordance with another embodiment of the invention, there is provided a physiological sensor system, comprising (i) means for measuring at least one physiological characteristic at a target measurement site on a subject's body, and (ii) means for heating an extended tissue region on the subject's body, whereby blood perfusion of the tissue region is enhanced, the extended tissue region including the target measurement site and a tissue region extending beyond the target measurement site.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a conventional pulse oximeter system;

FIGS. 2A and 2B are schematic illustrations of one embodiment of a pulse oximeter system with heating means, according to the invention;

FIG. 3 is a schematic illustration of the pulse oximeter system shown in FIG. 2B, showing heat applied to an appendage, i.e. arm and/or hand, of a subject and measurement of light absorption (i.e. oximeter reading) of the subject's heated finger, according to the invention;

FIG. 4 is a schematic illustration of the pulse oximeter system shown in FIG. 2B, showing heat applied to an ear of a subject and measurement of light absorption of the subject's heated ear, according to the invention;

FIGS. 5A and 5B are schematic illustrations of another embodiment of a pulse oximeter system having a plurality of sensors and associated heating means, according to the invention;

FIG. 6 is a schematic illustration of the pulse oximeter system shown in FIG. 5B, showing heat applied to an ear and arm of a subject and measurement of light absorption of the subject's heated ear and finger, according to the invention;

FIGS. 7 is an illustration of an IR portion of an oximetry plethysmogram obtained on an area of a subject's ear at a baseline temperature in the range of approximately 29-32° C., according to the invention;

FIGS. 8A and 8B are illustrations of IR portions of oximetry plethysmograms obtained on an area of the ear of first and second subjects, respectively, at an elevated temperature in the range of approximately 35-37° C., according to the invention; and

FIGS. 9 and 10 are graphical illustrations showing the effect of different heating method or conditions on pulse amplitude for subjects ranging in age from 71-94 years of age and 25-55 years of age, respectively, according to the invention.

### DETAILED DESCRIPTION OF THE INVENTION

Before describing the present invention in detail, it is to be understood that this invention is not limited to particularly exemplified methods or systems as such may, of course, vary. Thus, although a number of methods and systems similar or equivalent to those described herein can be used in the practice of the present invention, the preferred methods and systems are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### Definitions

The terms “pulse oximeter”, “oximeter sensor” and “oximeter”, as used herein, mean and include any conventional light-reflecting oximeter or sensor that is adapted to sense or measure light absorption in tissue and/or blood.

The term “oximeter reading”, as used herein, means and includes a measure of light absorption in tissue and/or blood.

The term “heating means”, as used herein, means and includes any means of increasing the core or tissue temperature of a subject, including, without limitation, one or more (i.e. a combination of) devices that transmit heat energy, such as thermoelectric heating devices (e.g., heating elements of various sizes, shapes, materials, etc. that are adapted to cooperate with various heating apparatus and/or configurations, such as a heated glove), contact heaters, lamps, heating blankets, etc., heated rooms, heated liquids, devices that transmit ultrasonic or photoelectric energy, and mentholated, counterirritant and/or vasodilating substances. The term “heating means” also means and includes passive heating means, i.e. means for limiting heat from escaping a specific tissue region of the body.

The terms “patient” and “subject”, as used herein, is meant to mean and include humans and animals.

The present invention substantially reduces or eliminates the disadvantages and drawbacks associated with conventional pulse oximetry methods and systems. In one embodiment of the invention, the pulse oximetry method and system includes an oximeter sensor and associated heating means that is adapted to heat a large tissue region or site, such as an entire organ or appendage, prior to or in conjunction with obtaining an oximeter reading. In another embodiment, the pulse oximetry method and system includes a plurality of oximeter sensors and associated heating means that are similarly adapted to selectively heat large tissue regions or sites prior to or in conjunction with obtaining oximeter readings.

As discussed in detail below, Applicants have found that the signal-to-noise ratio of a sensor, i.e. oximeter sensor, (and, hence, the accuracy of any physiological characteristic, e.g., O₂ saturation, determined therefrom) can be significantly enhanced by heating a significantly larger tissue region, i.e. a region that extends beyond the target measurement site and/or region in direct communication with the sensor, prior to or in conjunction with obtaining an oximeter reading.

Although the methods and systems of the invention are described herein in conjunction with pulse oximeter methods, sensors and systems, and measurements (or readings) obtained therewith, it is understood that the methods and systems are not limited to pulse oximetry and determinations made therefrom. Indeed, as will be appreciated by one having ordinary skill in the art, the methods and systems of the invention can readily be employed with other physiological monitoring apparatus and methods, which are adapted to monitor and/or determine a physiological characteristic based on the wave form, or amplitude or shape of a plethysmogram.

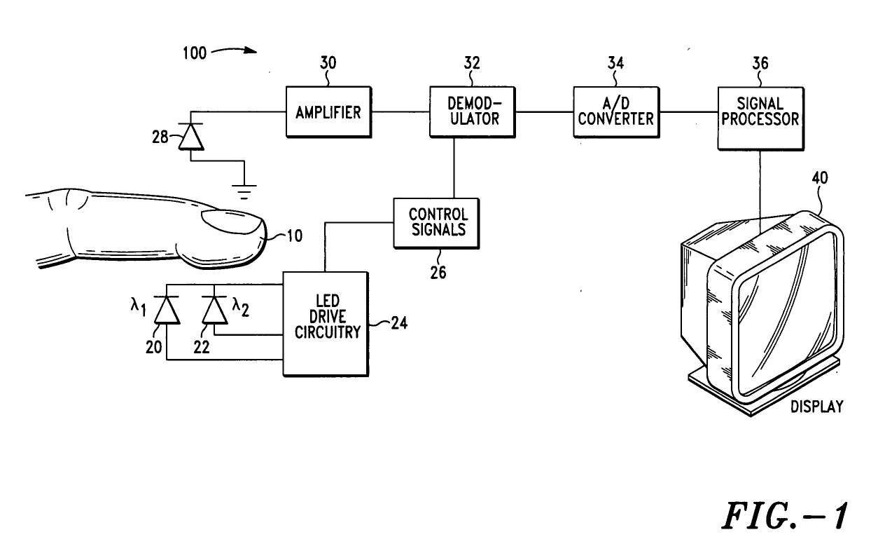

Referring first to FIG. 1, there is shown one embodiment of a conventional oximeter sensor and associated system (referred to hereinafter as “sensor” and denoted generally “100”) that can be employed within the scope of the present invention. As illustrated in FIG. 1, the sensor 100 preferably includes two emitters 20, 22 and detector 28, which are positioned adjacent the tissue being analyzed, i.e. finger 10.

Two lights are emitted by the emitters 20, 22; in one embodiment, a first light having a discrete wavelength in the range of approximately 650-670 nanometers in the red range and a second light having a discrete wavelength in the range of approximately 800-950 nanometers. The lights, in the illustrated embodiment, are transmitted through finger 10 via emitters 20, 22 and detected by detector 28.

The emitters 20, 22 are driven by drive circuitry 24, which is, in turn, governed by control signal circuitry 26. Detector 28 is in communication with or connected to amplifier 30. The signal from amplifier 30 is transmitted to demodulator 32, which is also synchronized to control signal circuitry 24. The demodulator 32, which is employed in most pulse oximeter systems, removes any common mode signals present and splits the time multiplexed signal into two (2) channels, one representing the red voltage (or optical) signal and the other representing the infrared voltage (or optical) signal.

The signal from the demodulator 32 is transmitted to an analog-digital converter 34. As is well known in the art, the output signal from the demodulator 34 is typically a time multiplexed signal comprising (i) a background signal, (ii) the red light range signal, and (iii) the infrared light range signal.

The desired computations are performed on the output from the converter 34 by signal processor 36 and the results transmitted to and displayed by display 40.

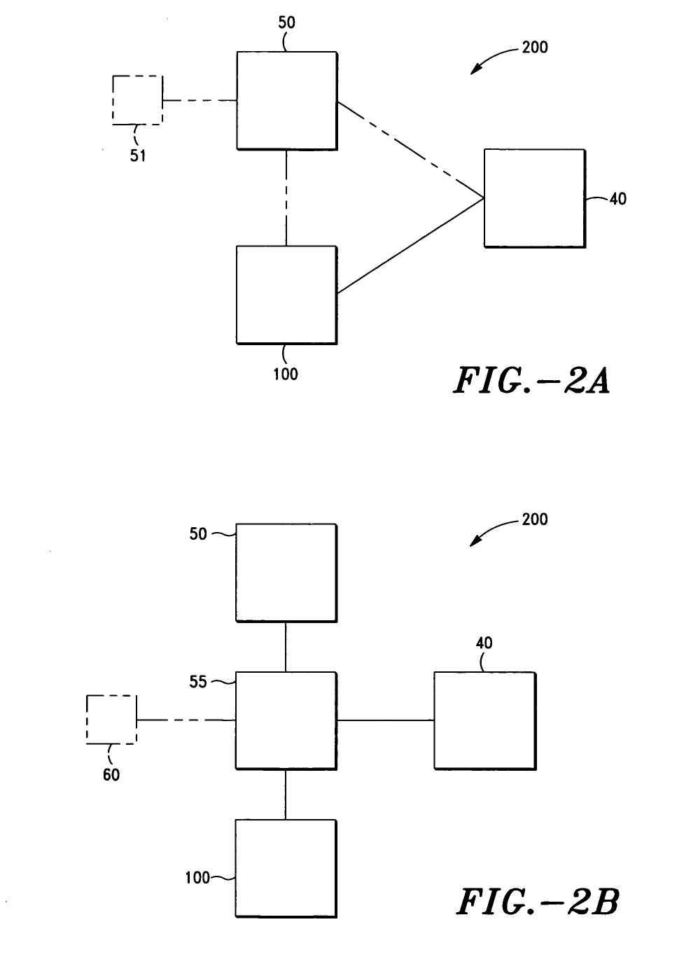

Referring now to FIG. 2A, there is shown a schematic illustration of one embodiment of a pulse oximeter system of the invention (denoted generally “200”). As illustrated in FIG. 2A, the system 200 includes sensor 100 (discussed above), heating means 50 and, optionally, display 40.

As will readily be appreciated by one having ordinary skill in the art. Various oximeter sensors (and systems) can be employed within the scope of the invention. Thus, although the pulse oximeter system 200 discussed in detail below employs sensor 100 (shown in FIG. 1), such use and discussion herein should not be deemed limiting.

Referring back to FIG. 2A, in some embodiments of the invention, the heating means 50 is connected to or in communication with, e.g., wireless communication, with sensor 100. Similarly, in some embodiments, heating means 50 is in communication with the display 40, whereby the heat transmitted by the heating means 50 can be displayed and, hence, monitored.

In some embodiments of the invention, the heating means 50 includes heat regulating means (shown in phantom and designated “51”), e.g., heating blanket, or integral control means, that is adapted to monitor and regulate the heat transmitted by the heating means 50.

Referring now to FIG. 2B, in some embodiments, the system 200 includes processor means (or processor) 55 that is in communication with heating means 50, sensor 100 and display 40, and is programmed and adapted to regulate heating means 50 and/or sensor 100 and/or the output displayed on display 40.

In yet additional embodiments, the system 200 further includes at least one heat sensor (shown in phantom and designated “60”) that is adapted to be disposed proximate the tissue region being heated by the heating means 50 and monitor the temperature of the heated tissue region. In the noted embodiments, the heat sensor 60 preferably is in communication with the processor 55 and, hence, display 40, whereby the temperature of the heated tissue region can be displayed.

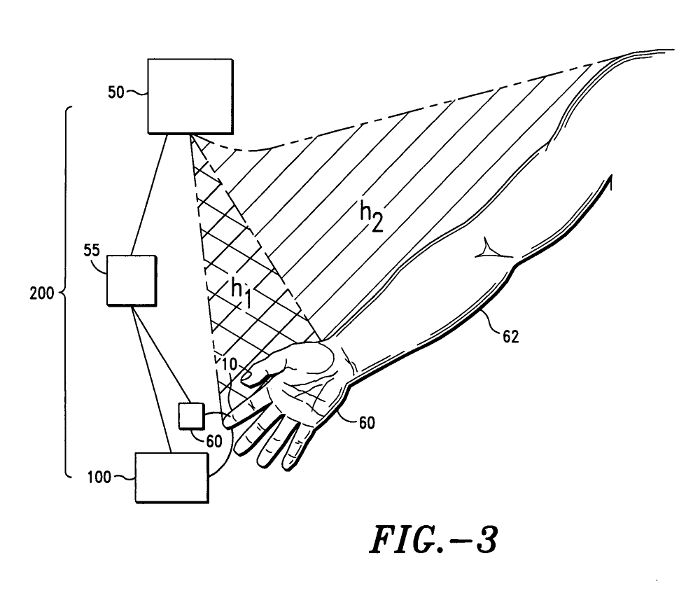

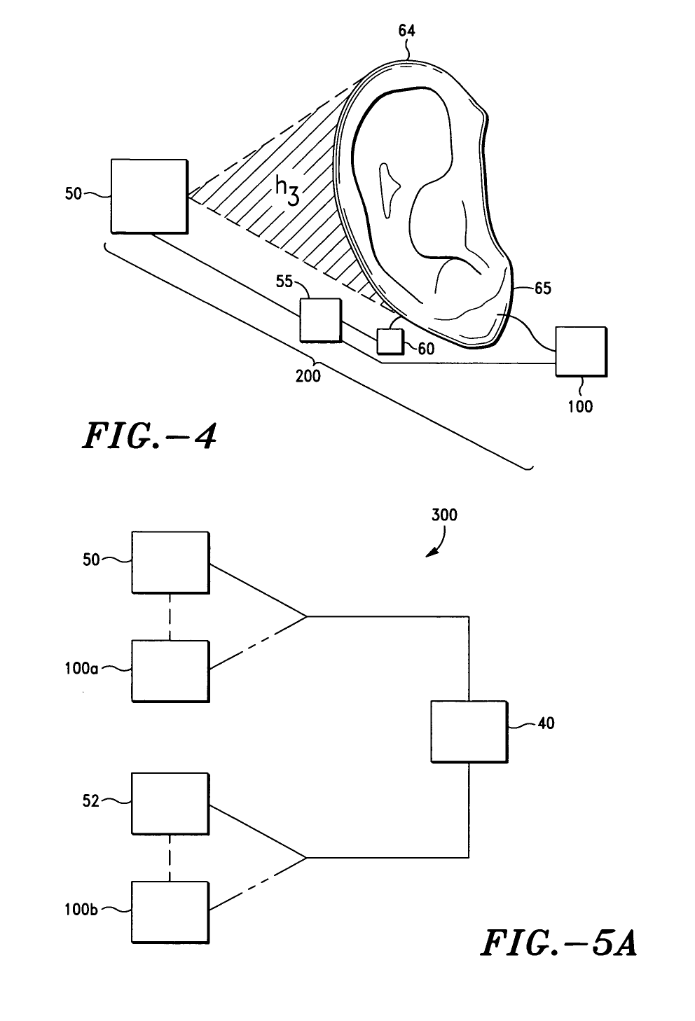

As indicated above, in a preferred embodiment of the invention, the heating means 50 of the invention is adapted to transmit heat energy to a large or extended tissue region, i.e. a tissue region that extends beyond the target measurement site and/or the tissue region that is proximate to or in direct communication with the sensor (see, e.g., FIGS. 3 and 4), prior to or in conjunction with obtaining an oximeter reading. In some embodiments of the invention, the heating means 50 is also adapted to heat a smaller tissue region, preferably, a tissue region proximate the sensor.

The heating means 50 of the invention can thus comprise any means of increasing the core or tissue (or skin) temperature of a subject, including, without limitation, devices that transmit heat energy, such as thermoelectric heating devices (e.g., heating elements of various sizes, shapes, materials, etc. that are adapted to cooperate with various heating apparatus and/or configurations, such as a heated glove), contact heaters, lamps, heating blankets, etc., heated rooms, heated liquids, devices that transmit ultrasonic or photoelectric energy, and mentholated, counterirritant and/or vasodilating substances, and passive heating means, i.e. means for limiting heat from escaping a specific tissue region of the body. As indicated above, the heating means 50 (and 52, discussed below) can also comprise two or more of the noted devices and means, e.g. two heat lamps.

According to the invention, the heat or heat energy provided by the heating means 50 can be substantially steady state (or constant) or varied, e.g. oscillated or any function of time-varied heating.

According to the invention, the heat or heat energy transmitted by the heating means 50 and applied to the tissue is sufficient to induce or trigger an optimal homeostatic reflex, whereby tissue perfusion of the heated tissue region is enhanced, without burning the patient. As will be appreciated my one having ordinary skill in the art, the amount of heat or heat energy that would be necessary to trigger an optimal homeostatic reflex will vary from patient-to-patient, site to site on the same patient as well as over time depending on physical and/or mental health condition, metabolic status, exertion or fatigue and prior thermal conditioning or exposure.

Applicants have, however, found that when the skin of a patient is heated up to a generally tolerable temperature range of approximately 40-42° C., arterioles in the blood vessel network that spread in the shallow layer within the dermis respond to the heat stimulus by active expansion of the inner diameters of the arterioles and general vasodialation. The expanded diameter results in a lowered resistance to blood flow and, hence, increased blood flow therethrough. Thus, in one embodiment of the invention, to optimize the increase of perfusion, the skin or tissue of the patient is heated to at least a temperature of approximately 35° C. or, at a minimum, 3° C. above the skin or surface temperature and below a temperature of approximately 42° C. to avoid burning the patient.

A key feature and advantage of the pulse oximeter methods and systems of the invention is the application of the heat or heat energy over a large tissue region, such as an entire organ or appendage, prior to or in conjunction with taking an oximeter reading. As indicated above, Applicants have found that the signal-to-noise ratio of an oximeter sensor (and, hence, the accuracy of any physiological characteristic, e.g., O₂ saturation, determined therefrom) can be significantly enhanced by heating a large tissue region prior to or in conjunction with obtaining an oximeter region. Indeed, Applicants have realized about one order of magnitude improvement in the signal-to-noise ratio by virtue of the methods and systems of the invention.

As will readily be appreciated by one having ordinary skill in the art, an order of magnitude increase in blood perfusion is significant in that the resulting signal strength enables measurement at an optimum site, such as a site proximate the central circulation, which is, by design, much less affected by vasoconstriction and, which is more proximal the heart and aorta. Such sites were heretofore deemed inaccessible and there was insufficient sensor signal strength to yield useful and high quality measurements, i.e. a quality that is comparable to conventional sites when non-constricted, such as the finger.

According to the invention, the large tissue region that is subjected to heating can, of course, comprise the entire body of the patient. The heating means 50, in this instance, could thus comprise a heated liquid bath or a heated room, such as a sauna.

More preferably, the larger tissue region comprises an entire organ or appendage and, in some embodiments, the adjoining tissue structure. Referring to FIG. 3, there is shown the application of heat to a hand 60 (shown as heat zone “h₁”) or alternatively, the entire arm 62 (shown as heat zone “h₂”) by heating means 50. According to the invention, the heat can be applied to the hand 60 and/or arm 62 prior to or in conjunction with obtaining an oximeter reading on a site therein, preferably, finger 10, with oximeter sensor 100.

In the noted illustration, the system 200 includes a heat sensor 60, which is disposed proximate the heated finger 10. However, as discussed in detail above, the heat sensor 60 can also be readily disposed proximate any desired location within heat zone “h₁” and, hence, hand 60 or heat zone “h₂” and, hence, arm 62. According to the invention, two or more heat sensors 60 can also be employed with system 200, e.g., one heat sensor 60 disposed proximate a location on the heated arm 62 and one heat sensor 60 disposed proximate the heated hand 60 or finger 10.

Referring to FIG. 4, there is shown the application of heat to an entire ear 64 by heating means 50 (shown as heat zone “h₃”). According to the invention, the heat applied to the ear 64 can be applied in such a manner (e.g., intensity and/or direction) that only a portion of the ear 64 is heated or the entire ear 64 is heated or the entire ear 64 and the adjoining tissue region or tissue and/or bone structure of the head are heated (unless otherwise stated, referred to collectively herein as “heated ear”). Thus, in one embodiment of the invention, a significant portion of the ear 64, more preferably, the entire ear 64 is heated. In another embodiment, the entire ear 64 and the adjoining tissue region or tissue and/or bone structure of the head (referred to collectively hereinafter as “adjoining tissue region) are heated.

According to the invention, the heat can similarly be applied to the ear 64 (or the entire ear 64 and the adjoining tissue region) prior to or in conjunction with obtaining an oximeter reading on a site therein, preferably, the earlobe 65, with oximeter sensor 100.

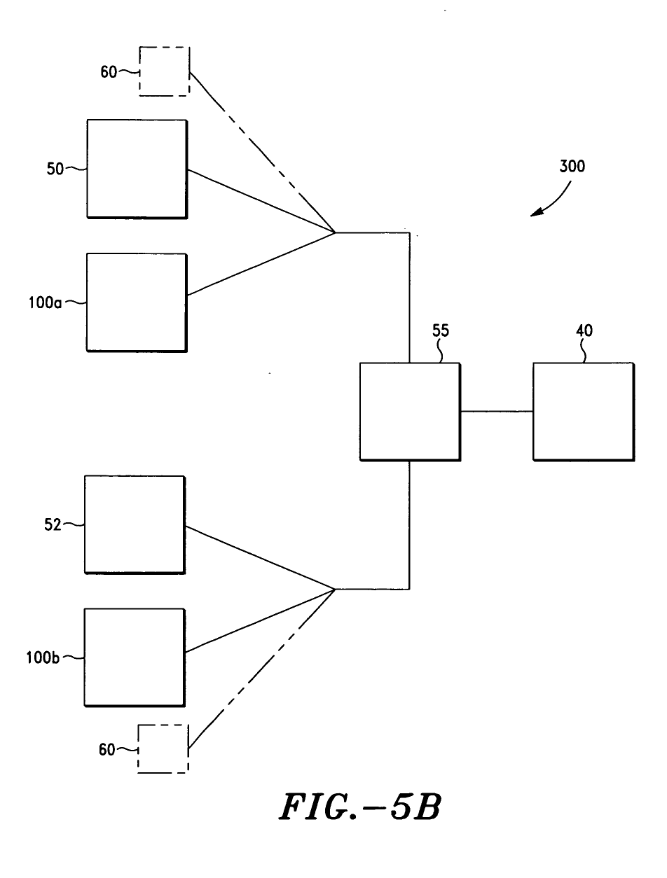

Referring now to FIG. 5A, there is shown a schematic illustration of another embodiment of a pulse oximeter method and system of the invention (denoted generally “300”). As illustrated in FIG. 5A, the system 300 includes a plurality of sensors 100a, 100b. According to the invention, the sensors 100a, 100b can be similar or comprise different sensors, e.g., different physical dimensions, attachment means, tuning, etc. Thus, in one embodiment of the invention, at least one sensor, i.e. 100a or 100b, is similar to sensor 100.

According to the invention, each sensor 100a, 100b is adapted to be positioned proximate to or on a desired position of the body, e.g., earlobe and finger, and obtain oximetry readings therefrom. In a preferred embodiment of the invention (discussed below), at least one sensor, e.g., 100a, is disposed proximate a central circulation site, e.g., neck, ear, nose, etc., and at least one sensor, e.g., 100b, is disposed proximate a peripheral circulation site, e.g., arm, hand, finger, etc.

The system 300 also includes a plurality of associated heating means 50, 52, which are similarly adapted to transmit heat energy to a large tissue region, i.e. a tissue region that extends beyond the respective sensor position or target measurement site and/or the tissue region that is proximate to or in direct communication with the respective sensor, prior to or in conjunction with obtaining an oximeter readings, and, optionally, display 40. The heating means 50, 52 are similarly adapted to be positioned proximate desired locations on the body and transmit heat or heat energy thereto; the term proximate meaning and including in close proximity to and/or in direct contact therewith.

As will be readily appreciated by one having ordinary skill in the art, each (or both) heating means 50, 52 of the invention can also be adapted to heat a smaller tissue region, e.g., a tissue region proximate a respective sensor, if desired.

According to the invention, heating means 52 can be similar to heating means 50, e.g., heat lamp, or, alternatively, heating means 50 and 52 can comprise different heat sources, e.g., heat lamp, heat blanket and passive heating means. As is also illustrated in FIG. 5A, each heating means 50, 52 can similarly be in communication with a respective sensor 100a, 100b and/or the display 40, whereby the heat transmitted by the heating means 50 and/or 52 can be displayed and, hence, monitored.

Although system 300 is shown with two sensors, i.e. sensors 100a, 100b, and associated heating means 50, 52, it is to be understood that system 300 can include more than two sensors with associated heating means, e.g. three, four, etc. The illustration of system 300 in FIGS. 5A (and 5B, discussed below) should thus not be deemed limiting in any manner.

Referring to FIG. 5B, in some embodiments, the system 300 similarly includes processor means (or processor) 55 that is in communication with heating means 50, 52,sensors 100a, 100b and display 40, and is programmed and adapted to regulate heating means 50, 52 and/or sensors 100a, 100b and/or the output displayed on display 40.

In yet additional embodiments, the system 300 further includes at least two heat sensors 60 that are similarly adapted to be disposed proximate the heated tissue regions and monitor the temperature thereof. In the noted embodiments, the heat sensors 60 are preferably in communication with the processor 55 and, hence, display 40, whereby the temperature of the heated tissue regions can be displayed.

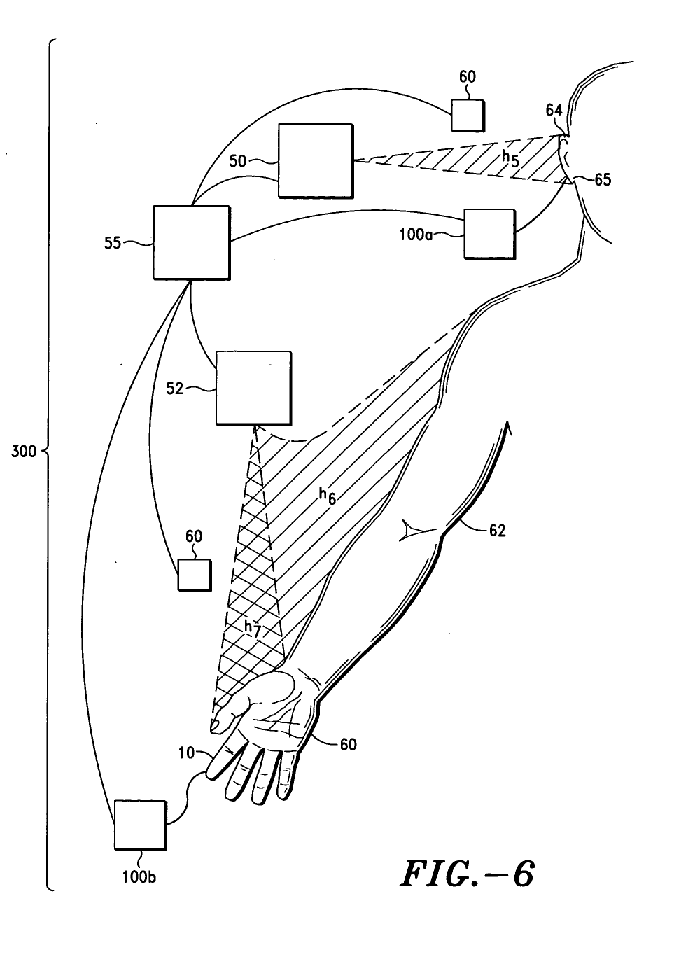

Referring now to FIG. 6, there is shown one application of system 300, where one sensor 100a is positioned proximate to and in communication with an earlobe 65 and one sensor 100b is positioned proximate to and in communication with a finger 10. As illustrated in FIG. 6, heating means 50 is also preferably positioned proximate the ear 64, where heating of the entire ear 64 (shown as heat zone “h₅”) or the ear 64 and adjoining tissue region is possible, if desired. Heating means 52 is preferably positioned proximate the arm 62 and hand 60, where heating of the arm 62 (shown as heat zone “h₆”) and/or hand 60 (shown as heat zone “h₇”) is possible, if desired.

According to the invention, one or both regions, e.g., ear 64 and arm 62, can be heated while obtaining oximetry readings with sensors 100a, 100b. Thus, in one embodiment of the invention, the entire ear 64 (or the ear 64 and adjoining tissue region) is heated with heating means 50 while oximeter readings are acquired at the heated earlobe 65 and the unheated finger 10 with sensors 100a and 100b, respectively. In another embodiment, the entire arm 62 is heated with heating means 52 while oximeter reading are acquired at the unheated earlobe 65 and heated finger 10 with sensors 100a and 100b, respectively. In yet another embodiment, the hand 60 is heated with heating means 52 while oximeter reading are acquired at the unheated earlobe 65 and finger 10 with sensors 100a and 100b, respectively. In yet another embodiment, the entire ear 64 (or the ear 64 and adjoining tissue region) is heated with heating means 50 and the hand 60 is heated with heating means 52 while oximeter reading are acquired at the heated earlobe 65 and heated finger 10 with sensors 100a and 100b, respectively.

According to the invention, oximetry readings can also be obtained with sensors 100a, 100b without the application of heat to an extended tissue region or during (or after a predetermined time after) the application of heat to a smaller tissue region proximate one or both sensors 100a, 100b.

System 300 thus provides an effective means of acquiring multiple oximetry readings with enhanced accuracy from sensors disposed at multiple locations on the body.

### EXAMPLES

The following examples are provided to enable those skilled in the art to more clearly understand and practice the present invention. They should not be considered as limiting the scope of the invention, but merely as being illustrated as representative thereof.

### Example 1

A series of blood oximetry readings were obtained from thirty-three (33) subjects that ranged in age from 28 to 92 years of age. Baseline temperature and plethysmographic readings were initially recorded. The baseline temperature for each subject was obtained on an area of the ear proximate the sensor using a remote IR skin temperature monitoring device. Baseline plethysmographic recordings were obtained with a non-heatable Nellcor Ear Sensor®, model ES-3212-9.

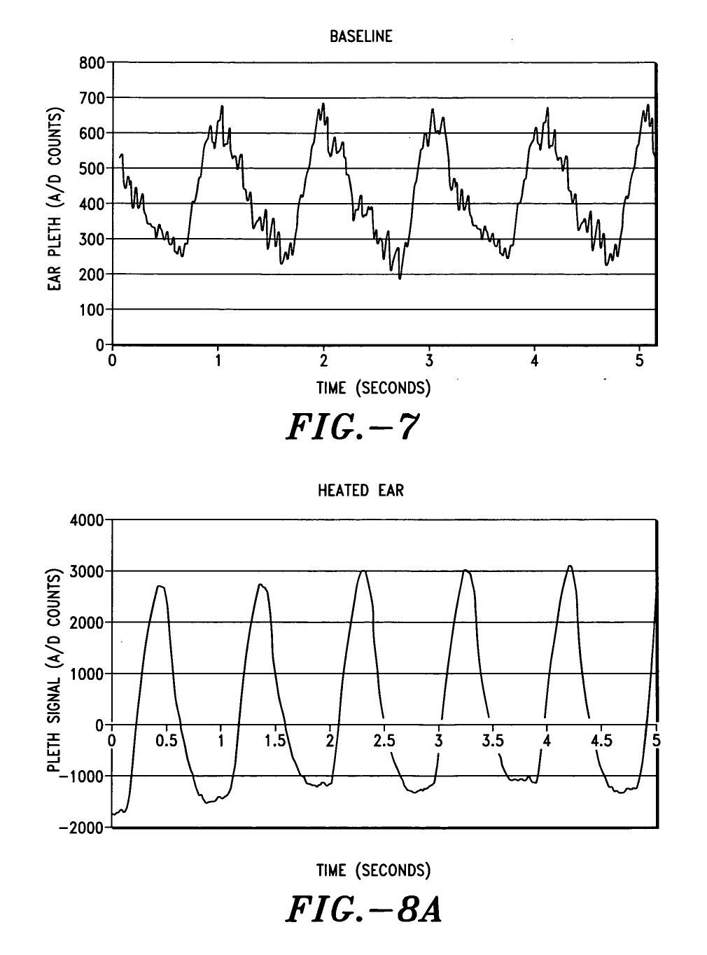

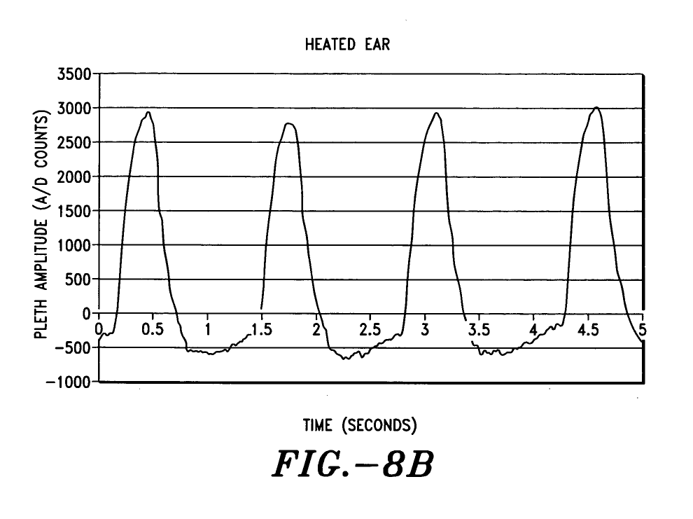

Referring now to FIGS. 7, 8A and 8B, there are shown the IR portions of oximetry plethysmograms obtained on an area of the ear at a baseline temperature in the range of approximately 29-32° C. (FIG. 7) and at an elevated temperature in the range of approximately 35-37° C. for two subjects (FIGS. 8A and 8B). It can be seen that the signal-to-noise ratio of the sensor is substantially improved in FIGS. 8B and 8B (i.e. elevated temperature), as evidenced by the absence of the spikes associated with the pulse waves at the baseline temperatures (i.e. FIG. 7).

It should further be noted that the amplitude of the pulse waves shown in FIG. 8A were increased from approximately 400 units (A/D counts) to approximately 3900 units, which reflects a substantial increase of approximately one order of magnitude.

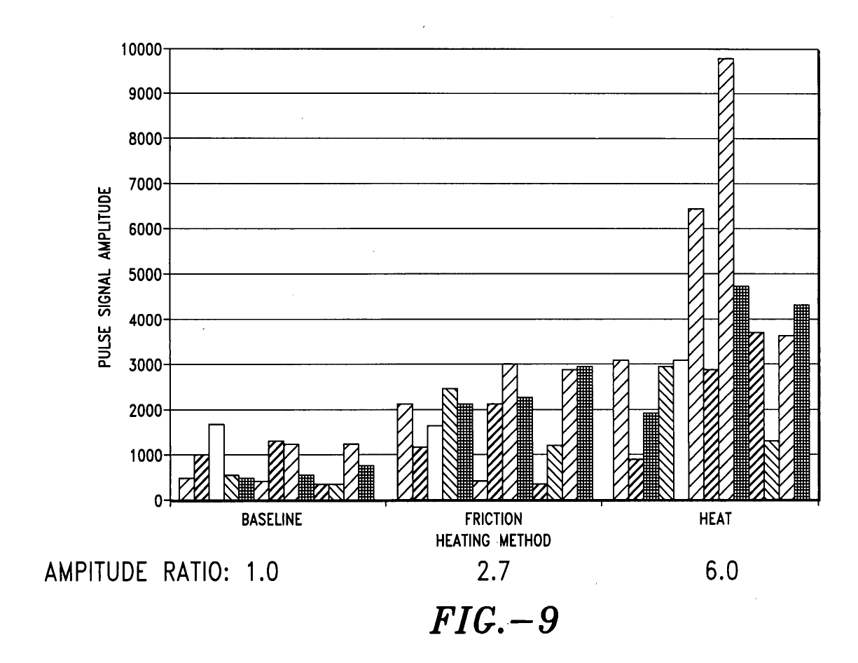

Referring now to FIG. 9, there is shown the effect of different heating methods or conditions for subjects ranging in age from 71-94 years of age on pulse amplitude (or signal). The heating methods or conditions comprised heating the ear to a temperature in the range of approximately 33-35° C. via “friction”, i.e. rubbing the earlobe for approximately 30 seconds, and active (or contact) heating, referred to as “heat” to a temperature of approximately 35-37° C. via a heater blanket.

As illustrated in FIG. 9, heating to a temperature of approximately 33-35° C. via “friction” produced an average 2.7-fold improvement in the amplitude ratio. Contact heating produced an average 6-fold improvement in the amplitude ratio.

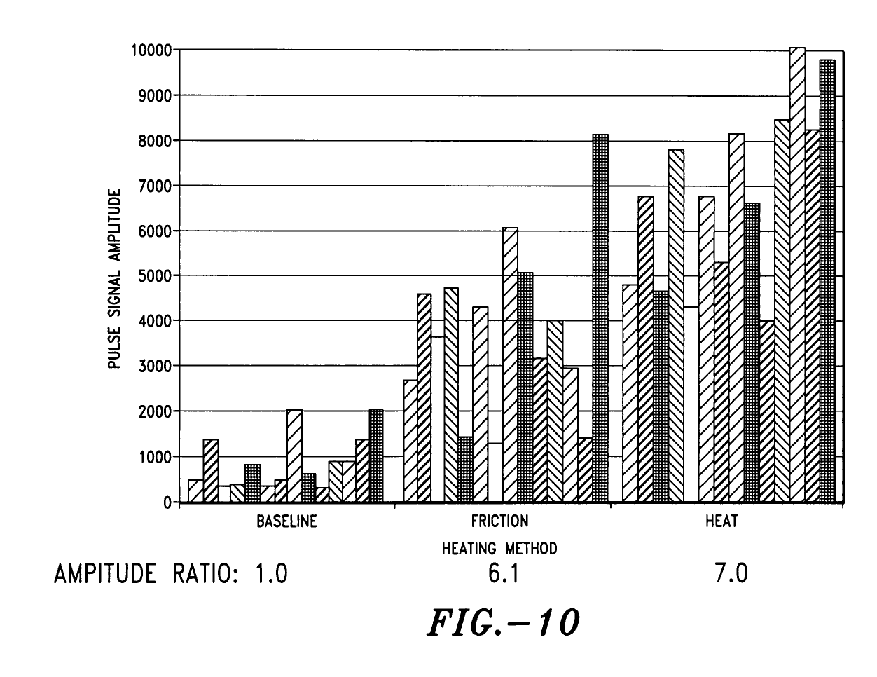

Referring now to FIG. 10, there is shown the effect of the same heating methods for subjects ranging in age from 25-26 years of age on the pulse amplitude. As illustrated in FIG. 10, “friction” heating produced an average 6.1-fold improvement in the amplitude ratio. Contact heating produced an average 10.7-fold improvement in the amplitude ratio.

The data reflected in FIGS. 7, 8A, 8B, 9 and 10 thus demonstrates that significant improvements in the signal-to-noise ratio of a sensor and, hence, the accuracy of physiological characteristics determined therefrom, can be obtained by virtue of the methods and systems of the invention.

As will readily be appreciated by one having ordinary skill in the art, the physiological sensor methods and systems of the invention provide numerous advantages. Among the advantages are the following:
The provision of physiological sensor methods and systems that enhance the accuracy of physiological measurements and determinations made therefrom.
The provision of pulse oximetry methods and systems that enhance the accuracy of blood parameter determinations of oximeter sensors, such as oxygen saturation.
The provision of pulse oximetry methods and systems that can readily be incorporated in or employed in conjunction with conventional oximeter sensors to enhance the accuracy of blood parameter readings and/or determinations made therefrom.
The provision of pulse oximetry methods and systems that facilitate the acquisition of signals reflecting physiological characteristic at a site that is supplied by the central circulation, such as a site on the head, and/or allows for monitoring of patients that are peripherally vasoconstricted to the extent that conventional sites, such as a finger or toe, are neither palpable, nor yield usable plethysmographic signals.
The provision of pulse oximetry methods and systems that facilitate the acquisition of signals reflecting physiological characteristic at a site that is proximate the aorta where the wave shape is much less influenced by transit through vasculature of complex shape, branching and length at a patient-dependent degree of hardening of the arterial wall. Thus, the pressure and flow wave shape is more similar to the original shape as it leaves the aorta, which enables accurate measurements and diagnostic information of hemodynamic parameters, such as blood pressure, cardiac output, structure condition and functioning of the arterial vasculature.
The provision of pulse oximetry methods and systems that provide heating at a constant or variable rate to a set temperature and monitoring of amplitudes or time changes of the arterial pressure induced signals, whereby the pressure or flow waveforms yields information on the degree of physiological control of that patient, as well as indirectly on therapeutic or otherwise interventional effectiveness.
The provision of pulse oximetry methods and systems that include thermal control of the measurement site and sensing system, whereby accurate data is provided that is not affected by temperature variability or fluctuation.

Without departing from the spirit and scope of this invention, one having ordinary skill in the art can make various changes and modifications to the invention to adapt it to various usages and conditions. As such, these changes and modifications are properly, equitably, and intended to be, within the full range of equivalence of the following claims.

What is claimed is:

1. A method of determining a physiological characteristic, comprising the steps of:
providing at least one physiological sensor that is adapted to measure at least one physiological characteristic at a target measurement site on a subject's body;
disposing said physiological sensor proximate said target measurement site on said subject's body;
heating a tissue region on said subject's body, whereby blood perfusion of said tissue region is enhanced, said tissue region including said target measurement site and extending beyond said target measurement site; and
measuring at least one physiological physiological characteristic at said target measurement site with said physiological sensor during or within a predetermined period after said heating of said tissue region.

2. The method of claim 1, wherein said heating of said tissue region is sufficient to induce an optimal homeostatic reflex, whereby said tissue region blood perfusion is enhanced, without burning said subject.

3. The method of claim 1, wherein said tissue region comprises the entire ear of said subject and said target measurement site comprises the earlobe of said heated ear.

4. The method of claim 1, wherein said tissue region comprises the entire ear and adjoining tissue region of said subject and said target measurement site comprises the earlobe of said heated ear.

5. The method of claim 1, wherein said tissue region comprises the entire arm of said subject and said target measurement site comprises a finger on said heated arm.

6. The method of claim 1, wherein said tissue region comprises a hand of said subject and said target measurement site comprises a finger on said heated hand.

7. A method of determining a physiological characteristic, comprising the steps of:
providing a plurality of physiological sensors that are adapted to measure at least one physiological characteristic at target measurement sites on a subject's body;
disposing a first physiological sensor proximate a first target measurement site on said subject's body;
disposing a second physiological sensor proximate a second target measurement site on said subject's body;
heating a first tissue region on said subject's body, whereby blood perfusion of said first tissue region is enhanced, said first tissue region including said first target measurement site and extending beyond said first target measurement site; and
measuring at least one physiological characteristic with said first and second physiological sensors during or within a predetermined period after said heating of said first tissue region.

8. The method of claim 7, wherein said heating of said first tissue region is sufficient to induce an optimal homeostatic reflex, whereby said first tissue region blood perfusion is enhanced, without burning said subject.

9. The method of claim 7, wherein said first tissue region comprises the entire ear of said subject and said first target measurement site comprises the earlobe of said heated ear.

10. The method of claim 7, wherein said first tissue region comprises the entire ear and adjoining tissue region of said subject and said first target measurement site comprises the earlobe of said heated ear.

11. The method of claim 7, wherein said first tissue region comprises the entire arm of said subject and said first target measurement site comprises a finger on said heated arm.

12. The method of claim 7, wherein said first tissue region comprises a hand of said subject and said first target measurement site comprises a finger on said heated hand.

13. A method of determining a physiological characteristic, comprising the steps of:
providing a plurality of physiological sensors that are adapted to measure at least one physiological characteristic at target measurement sites on a subject's body;
disposing a first physiological sensor proximate a first target measurement site on said subject's body;
disposing a second physiological sensor proximate a second target measurement site on said subject's body;
heating a first tissue region on said subject's body, whereby blood perfusion of said first tissue region is enhanced, said first tissue region including said first target measurement site and extending beyond said first target measurement site;
heating a second tissue region on said subject's body, whereby blood perfusion of said second tissue region is enhanced, said second tissue region including said second target measurement site and extending beyond said second target measurement site; and
measuring at least one physiological characteristic with said first and second physiological sensors during or within a predetermined period after said heating of said first and second tissue regions.

14. The method of claim 13, wherein said heating of said first tissue region is sufficient to induce an optimal homeostatic reflex proximate said first tissue region, whereby said first tissue region blood perfusion is enhanced, without burning said subject.

15. The method of claim 13, wherein said heating of said second tissue region is sufficient to induce an optimal homeostatic reflex proximate said second tissue region, whereby said second tissue region blood perfusion is enhanced, without burning said subject.

16. The method of claim 13, wherein said first tissue region comprises the entire ear of said subject and said first target measurement site comprises the earlobe of said heated ear.

17. The method of claim 1, wherein said first tissue region comprises the entire ear and adjoining tissue region of said subject and said first target measurement site comprises the earlobe of said heated ear.

18. The method of claim 17, wherein said second tissue region comprises the entire arm of said subject and said second target measurement site comprises a finger on said heated arm.

19. A physiological sensor system, comprising:
means for measuring at least one physiological characteristic at a target measurement site on a subject's body; and
means for heating a tissue region on said subject's body, whereby blood perfusion of said tissue region is enhanced, said tissue region including said target measurement site and extending beyond said target measurement site.
